(19) United States
(12) Patent Application Publication
(10) Pub. No.: US 2024/0174982 A1
(43) Pub. Date: May 30, 2024

(54) METHODS OF PRODUCING THREE-DIMENSIONAL CELLULAR TISSUES, AND THREE-DIMENSIONAL CELLULAR TISSUES

(71) Applicant: TOPPAN Inc., Tokyo (JP)

(72) Inventors: Shiro KITANO, Taito-ku (JP); Kei TSUKAMOTO, Taito-ku (JP); Yuma YOKOKAWA, Taito-ku (JP)

(73) Assignee: TOPPAN Inc., Tokyo (JP)

(21) Appl. No.: 18/437,415

(22) Filed: Feb. 9, 2024

Related U.S. Application Data
(63) Continuation of application No. PCT/JP2022/030224, filed on Aug. 8, 2022.

(30) Foreign Application Priority Data
Aug. 11, 2021 (JP) 2021-131168

(51) Int. Cl. C12N 5/077; C12N 5/09

(52) U.S. Cl. CPC C12N 5/0669; C12N 5/0693; C12N 2501/90; C12N 2501/998; C12N 2513/00; C12N 2533/56; C12N 2533/76

(57) ABSTRACT

A method of producing three-dimensional cellular tissues includes: obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; gelling the stromal cell-containing mixture to obtain a first gel composition containing stromal cells; obtaining a target cell-containing mixture containing target cells, a cationic substance, an extracellular matrix component and a polyelectrolyte, or a target cell-containing mixture containing target cells, a cationic substance and a fragmented extracellular matrix component; placing the target cell-containing mixture in contact with the first gel composition; gelling the target cell-containing mixture to obtain a second gel composition containing target cells; and incubating the first gel composition and the second gel composition to obtain three-dimensional cellular tissues.

### CROSS-REFERENCE TO RELATED APPLICATIONS

The present application is a continuation of and claims the benefit of priority to International Application No. PCT/JP2022/030224, filed Aug. 8, 2022, which is based upon and claims the benefit of priority to Japanese Application No. 2021-131168, filed Aug. 11, 2021. The entire contents of these applications are incorporated herein by reference.

### BACKGROUND OF THE INVENTION

### Field of the Invention

The present invention relates to methods of producing three-dimensional cellular tissues, and three-dimensional cellular tissues.

### Description of Background Art

JP 6639634 B describes a technique for producing three-dimensional cellular tissues, including obtaining a mixture containing cells suspended in a solution containing at least a cationic buffer solution, an extracellular matrix component and a polyelectrolyte, collecting the cells from the obtained mixture to form cell aggregates on a substrate, and culturing the cells to obtain three-dimensional cellular tissues. The entire contents of this publication are incorporated herein by reference.

### SUMMARY OF THE INVENTION

According to one aspect of the present invention, a method of producing three-dimensional cellular tissues includes obtaining a stromal cell-containing mixture including stromal cells, a cationic substance, an extracellular matrix component and a polyelectrolyte, or a stromal cell-containing mixture including stromal cells, a cationic substance and a fragmented extracellular matrix component, gelling the stromal cell-containing mixture to obtain a first gel composition including stromal cells, obtaining a target cell-containing mixture including target cells, a cationic substance, an extracellular matrix component and a polyelectrolyte, or a target cell-containing mixture including target cells, a cationic substance and a fragmented extracellular matrix component, placing the target cell-containing mixture in contact with the first gel composition, gelling the target cell-containing mixture to obtain a second gel composition including target cells, and incubating the first gel composition and the second gel composition to obtain three-dimensional cellular tissues.

According to another aspect of the present invention, a three-dimensional cellular tissue in which a target cell gel section including target cells, a cationic substance, an extracellular matrix component, a polyelectrolyte and a second gel component, or a target cell gel section including target cells, a cationic substance, a fragmented extracellular matrix component and a second gel component is placed in contact with a stromal cell gel section including stromal cells, a cationic substance, an extracellular matrix component, a polyelectrolyte and a first gel component, or a stromal cell gel section including stromal cells, a cationic substance, a fragmented extracellular matrix component and a first gel component.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a representative micrograph showing the positions of cancer cells in a three-dimensional cellular tissue as determined in Experimental Example 2;

FIG. 2 is representative micrographs of three-dimensional cellular tissue fragments on day 1 and day 8 of culture as determined in Experimental Example 2;

FIG. 3A is a representative micrograph of a three-dimensional cellular tissue on day 8 of culture, in which cancer cells are stained, as determined in Experimental Example 3;

FIG. 3B is a representative micrograph of a three-dimensional cellular tissue on day 8 of culture, in which stromal cells are stained, as determined in Experimental Example 3; and

FIG. 4 is representative micrographs of three-dimensional cellular tissue fragments on day 1 and day 8 of culture as determined in Experimental Example 4.

### DETAILED DESCRIPTION OF THE EMBODIMENTS

Embodiments will now be described with reference to the accompanying drawings, wherein like reference numerals designate corresponding or identical elements throughout the various drawings.

### Method of Producing Three-Dimensional Cellular Tissues

A method of producing three-dimensional cellular tissues according to one embodiment of the present invention includes obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; gelling the stromal cell-containing mixture to obtain a first gel composition containing stromal cells; obtaining a target cell-containing mixture containing target cells, a cationic substance, an extracellular matrix component and a polyelectrolyte, or a target cell-containing mixture containing target cells, a cationic substance and a fragmented extracellular matrix component; placing the target cell-containing mixture in contact with the first gel composition; gelling the target cell-containing mixture to obtain a second gel composition containing target cells; and incubating the first gel composition and the second gel composition to obtain three-dimensional cellular tissues.

According to the production method of the present embodiment, it is possible to maintain target cells at predetermined positions in a three-dimensional cellular tissue. This makes it possible to construct an organ that requires more structural three-dimensional control. In addition, since the thickness of the target cells can be maintained, a large tissue can be cultured for a long period of time. Furthermore, since the three-dimensional cellular tissues obtained by the production method of the present embodiment can be used to easily evaluate the size, degree of migration, and the like of the mass of the target cells, they can be used to easily evaluate, for example, sensitivity to anticancer drugs, metastasis of cancer cells, degree of invasion of cancer cells, and the like, and thus can be used for screening anticancer drugs.

The term “three-dimensional cellular tissue” as used herein refers to a three-dimensional cell aggregate. The uses of three-dimensional cellular tissues include, but are not limited to, biological tissue models and solid cancer models. Examples of the biological tissue models include skin, hair, bone, cartilage, tooth, cornea, blood vessel, lymphatic vessel, heart, liver, pancreas, nerve and esophagus models. Examples of the solid cancer models include gastric cancer, esophageal cancer, colorectal cancer, colon cancer, rectal cancer, pancreatic cancer, breast cancer, ovarian cancer, prostate cancer, renal cell cancer and liver cancer models.

Further, the form of the three-dimensional cellular tissue is not particularly limited, and may be, for example, a three-dimensional cellular tissue formed by culturing cells in a vessel such as a cell culture insert. The form of the three-dimensional cellular tissue may be a three-dimensional cellular tissue formed by culturing cells within a scaffold composed of a natural biopolymer, such as collagen, or a synthetic polymer. The form of the three-dimensional cellular tissue may be a cell aggregate (spheroid) or may be a sheet-like cell structure.

A method of producing three-dimensional cellular tissues according to the present embodiment includes: (a) obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; (b) gelling the stromal cell-containing mixture to obtain a first gel composition containing stromal cells; (c) obtaining a target cell-containing mixture containing target cells, a cationic substance, an extracellular matrix component and a polyelectrolyte, or a target cell-containing mixture containing target cells, a cationic substance and a fragmented extracellular matrix component; (d) placing the target cell-containing mixture in contact with the first gel composition; (e) gelling the target cell-containing mixture to obtain a second gel composition containing target cells; and (f) incubating the first gel composition and the second gel composition to obtain three-dimensional cellular tissues. Each process will be described below.

First, in process (a), a stromal cell-containing mixture containing stromal cells, a cationic substance, an extracellular matrix component and a polyelectrolyte, or a stromal cell-containing mixture containing stromal cells, a cationic substance and a fragmented extracellular matrix component is obtained. The mixing of the stromal cells, the cationic substance, the extracellular matrix component and the polyelectrolyte or the mixing of the stromal cells, the cationic substance and the fragmented extracellular matrix component may be performed in an aqueous solvent. Examples of the aqueous solvent include, but are not limited to, water, buffer solutions and media.

### Stromal Cells

The term “stromal cells” as used herein refers to cells constituting supporting tissues of epithelial cells. Example of the stromal cells used in the present embodiment include fibroblasts, immune cells, vascular endothelial cells and smooth muscle cells. Examples of the immune cells include lymphocytes, neutrophils and macrophages. Stromal cells are essential for maintenance of tissues, and play important roles in inflammatory responses, wound healing responses, and the like. In the present invention, the stromal cells can maintain the target cells.

The origin of the stromal cells is not particularly limited, and cells derived from mammals such as humans, monkeys, dogs, cats, rabbits, pigs, cows, mice and rats can be used.

### Cationic Substance

As the cationic substance, any substance having a positive charge can be used as long as it does not adversely affect the growth of stromal cells and formation of stromal cell aggregate described later. Examples of the cationic substance include, but are not limited to, cationic buffers, such as tris-hydrochloric acid, tris-maleic acid, bis-tris and HEPES, ethanolamine, diethanolamine, triethanolamine, polyvinylamine, polyallylamine, polylysine, polyhistidine, polyarginine, and the like. In particular, a cationic buffer is preferred, and tris-hydrochloric acid buffer is more preferred.

The concentration of the cationic substance in the stromal cell-containing mixture in process (a) is not particularly limited as long as it does not adversely affect the growth of stromal cells and formation of stromal cell aggregate. The concentration of the cationic substance used in the present embodiment is preferably 10 mM to 100 mM relative to the volume of the aqueous solvent, more preferably 20 mM to 90 mM, still more preferably 30 mM to 80 mM, still even more preferably 40 mM to 70 mM, and yet still even more preferably 45 mM to 60 mM.

When a cationic buffer is used as the cationic substance, the pH of the cationic buffer solution is not particularly limited as long as it does not adversely affect the growth of stromal cells and formation of stromal cell aggregate. The pH of the cationic buffer solution used in the present embodiment is preferably 6.0 to 8.0. For example, the pH of the cationic buffer solution used in the present embodiment may be 7.0, 7.1, 7.2, 7.3, 7.4, 7.5, 7.6, 7.7, 7.8, 7.9 or 8.0. The pH of the cationic buffer solution used in the present embodiment is more preferably 7.2 to 7.6, and still more preferably approximately 7.4.

### Extracellular Matrix Component

As the extracellular matrix component, any component constituting an extracellular matrix (ECM) can be used as long as it does not adversely affect the growth of stromal cells and formation of stromal cell aggregate described later. Examples of the extracellular matrix component include, but are not limited to, collagen, laminin, fibronectin, vitronectin, elastin, tenascin, entactin, fibrillin, proteoglycan, and combinations thereof. Examples of the extracellular matrix component may further include modified forms and variants of the above components. These extracellular matrix components may be used singly or in combination of two or more.

Examples of the proteoglycan include chondroitin sulfate proteoglycan, heparan sulfate proteoglycan, keratan sulfate proteoglycan and dermatan sulfate proteoglycan. In particular, collagen, laminin and fibronectin are preferred, and collagen is particularly preferred as the extracellular matrix component.

The total content of the extracellular matrix component in the mixture in process (a) is not particularly limited as long as it does not adversely affect the growth of stromal cells and formation of stromal cell aggregate, and may be 0.005 mg/mL or more and 1.5 mg/mL or less, preferably 0.005 mg/mL or more and 1.0 mg/mL or less, more preferably 0.01 mg/mL or more and 1.0 mg/mL or less, still more preferably 0.025 mg/mL or more and 1.0 mg/mL or less, and still even more preferably 0.025 mg/mL or more and 0.1 mg/mL or less. The extracellular matrix component may be dissolved in an appropriate solvent before use. Examples of the solvent include, but are not limited to, water, buffer solutions and acetic acid. In particular, buffer solutions or acetic acid is preferred.

### Polyelectrolyte

The term “polyelectrolyte” as used herein refers to a polymer having a dissociable functional group in the polymer chain. As the polyelectrolyte used in the present embodiment, any polyelectrolyte can be used as long as it does not adversely affect the growth of stromal cells and formation of stromal cell aggregate. Examples of the polyelectrolyte include, but are not limited to, glycosaminoglycans such as heparin, chondroitin sulfate (e.g., chondroitin 4-sulfate, or chondroitin 6-sulfate), heparan sulfate, dermatan sulfate, keratan sulfate and hyaluronic acid; dextran sulfate, rhamnan sulfate, fucoidan, carrageenan, polystyrene sulfonic acid, polyacrylamide-2-methylpropanesulfonic acid, polyacrylic acid, and combinations thereof. The polyelectrolyte may be derivatives of those described above. These polyelectrolytes may be used singly or in combination of two or more.

The polyelectrolyte is preferably a glycosaminoglycan. In particular, heparin, chondroitin sulfate and dermatan sulfate are preferred, and heparin is particularly preferred.

The concentration of the polyelectrolyte in the mixture in process (a) is not particularly limited as long as it does not adversely affect the growth of stromal cells and formation of stromal cell aggregate. Unlike the extracellular matrix component, the polyelectrolyte is effective at any concentration as long as it is below the solubility limit, and does not inhibit the effects of the extracellular matrix component. The concentration of polyelectrolyte is preferably 0.005 mg/mL or more, and preferably 0.005 mg/mL or more and 1.0 mg/mL or less, more preferably 0.01 mg/mL or more and 1.0 mg/mL or less, still more preferably 0.025 mg/mL or more and 1.0 mg/mL or less, and still even more preferably 0.025 mg/mL or more and 0.1 mg/mL or less.

The polyelectrolyte may be dissolved in an appropriate solvent before use. Examples of the solvent include, but are not limited to, water and buffer solutions. When a cationic buffer solution is used as the above cationic substance, the polyelectrolyte may be dissolved in a cationic buffer solution before use.

A mixing ratio (final concentration ratio) between the polyelectrolyte and the extracellular matrix component in the stromal cell-containing mixture in process (a) is preferably 1:2 to 2:1, more preferably 1:1.5 to 1.5:1, and still more preferably 1:1.

### Fragmented Extracellular Matrix Component

In process (a), an extracellular matrix component that is fragmented (hereinafter, also referred to as a fragmented extracellular matrix component) may be used instead of the extracellular matrix component and the polyelectrolyte. That is, in process (a), the stromal cell-containing mixture may contain stromal cells, a cationic substance and a fragmented extracellular matrix component. Further, the stromal cell-containing mixture may contain a fragmented extracellular matrix component in addition to the extracellular matrix component and the polyelectrolyte. That is, in process (a), the stromal cell-containing mixture may contain stromal cells, a cationic substance, an extracellular matrix component, a polyelectrolyte and a fragmented extracellular matrix component.

The fragmented extracellular matrix component can be obtained by fragmenting the above extracellular matrix component. The “fragmentation” means reducing an aggregate of extracellular matrix molecules to a smaller size. Fragmentation may be performed under conditions that cleave the bonds in extracellular matrix molecules, or may be performed under conditions that do not cleave the bonds in extracellular matrix molecules. The fragmented extracellular matrix component may contain a defibrated extracellular matrix component, which is a component obtained by defibrating the above extracellular matrix component by applying a physical force. Defibration is a form of fragmentation, and may be performed under conditions, for example, that do not cleave the bonds in extracellular matrix molecules.

The method for fragmenting an extracellular matrix component is not particularly limited. As the method for defibrating an extracellular matrix component, a physical force may be applied with an ultrasonic homogenizer, a stirring homogenizer, a high pressure homogenizer, or the like to defibrate the extracellular matrix component. When using a stirring homogenizer, the extracellular matrix component may be homogenized as it is, or may be homogenized in an aqueous medium such as saline. Further, it is also possible to obtain a millimeter-sized or nanometer-sized defibrated extracellular matrix component by adjusting the time, number of times, and the like of homogenization. Also, a defibrated extracellular matrix component can be obtained by defibration through repeated freezing and thawing.

The fragmented extracellular matrix component may contain, at least in part, a defibrated extracellular matrix component. Alternatively, the fragmented extracellular matrix component may be composed of only a defibrated extracellular matrix component. That is, the fragmented extracellular matrix component may be a defibrated extracellular matrix component. The defibrated extracellular matrix component preferably contains a collagen component that is defibrated (also referred to as a defibrated collagen component). The defibrated collagen component preferably maintains a triple-helical structure derived from collagen. When dispersed in an aqueous medium, the fragmented collagen component can easily come into contact with cells in the aqueous medium, promoting formation of three-dimensional cellular tissues.

The fragmented extracellular matrix component may have a fibrous shape, for example. The “fibrous” means a shape formed of fiber-like extracellular matrix components or a shape formed of fiber-like extracellular matrix components crosslinked between molecules. At least part of the fragmented extracellular matrix component may be fibrous. The fibrous extracellular matrix component further includes a fine fiber-like substance formed by aggregation of multiple fiber-like extracellular matrix molecules (fibrils), a fiber-like substance formed of an aggregation of fibrils, defibrated fiber-like substances, and the like. In the fibrous extracellular matrix component, the RGD sequence is preserved without being destroyed and can further effectively function as a scaffold for cell adhesion.

The average length of the fragmented extracellular matrix component may be 100 nm or more and 400 μm or less, and preferably 100 nm or more and 200 μm or less. In one embodiment, from the viewpoint of facilitating thick tissue formation, the average length of the fragmented extracellular matrix component may be 5 μm or more and 400 μm or less, preferably 10 μm or more and 400 μm or less, more preferably 22 μm or more and 400 μm or less, and still more preferably 100 μm or more and 400 μm or less. In another embodiment, from the viewpoint of facilitating stable tissue formation and improving redispersibility, the average length of the fragmented extracellular matrix component may be 100 nm or more and 100 μm or less, preferably 100 nm or more and 50 μm or less, more preferably 100 nm or more and 30 μm or less, still more preferably 100 nm or more and 15 μm or less, still even more preferably 100 nm or more and 10 μm or less, and yet still even more preferably 100 nm or more and 1 μm or less. It is preferred that, among the entire fragmented extracellular matrix component, most part of the fragmented extracellular matrix component has the average length within the above numerical range. Specifically, it is preferred that 95% of the entire fragmented extracellular matrix component has the average length within the above numerical range. The fragmented extracellular matrix component is preferably a fragmented collagen component having the average length within the above range, and more preferably a defibrated collagen component having the average length within the above range.

The average diameter of the fragmented extracellular matrix component may be in a range of 50 nm to 30 μm, preferably 4 μm to 30 μm, and more preferably 5 μm to 30 μm. The fragmented extracellular matrix component is preferably a fragmented collagen component having the average diameter within the above range, and more preferably a defibrated collagen component having the average diameter within the above range.

Since the above ranges of the average length and diameter are optimized from the viewpoint of three-dimensional cellular tissue formation, it is preferred that the fragmented extracellular matrix component has the average length and diameter within the above range when resuspended in an aqueous medium for tissue formation after a drying process, which will be described later.

The average length and diameter of the fragmented extracellular matrix component can be determined by measuring individual pieces of the fragmented extracellular matrix component with an optical microscope and performing image analysis. The term “average length” as used herein refers to the average length of the measured samples in the longitudinal direction, and the term “average diameter” refers to the average length of the measured samples in the direction perpendicular to the longitudinal direction.

At least part of the fragmented extracellular matrix component may be crosslinked intermolecularly or intramolecularly. The fragmented extracellular matrix component may be crosslinked within molecules constituting the fragmented extracellular matrix component, or may be crosslinked between molecules constituting the fragmented extracellular matrix component.

Examples of the crosslinking method include, but are not particularly limited to, physical crosslinking by applying heat, ultraviolet light, radiation, or the like, and chemical crosslinking using crosslinking agents, enzyme reactions, or the like. The crosslinking (that is, physical crosslinking and chemical crosslinking) may be crosslinking via covalent bonds.

When the extracellular matrix component contains a collagen component, crosslinks may be formed between triple-helical structures of collagen molecules or may be formed between collagen fibrils formed of collagen molecules. The crosslinking may be crosslinking by heat (that is, thermal crosslinking). The thermal crosslinking may be performed by, for example, applying heat treatment under reduced pressure using a vacuum pump. In thermal crosslinking of a collagen component, the extracellular matrix component may be crosslinked by forming peptide bonds (—NH—CO—) between the amino group of collagen molecules and the carboxy group of the same or other collagen molecules.

The extracellular matrix component can also be crosslinked using a crosslinking agent. The crosslinking agent may be, for example, one capable of crosslinking carboxyl groups and amino groups, or capable of crosslinking amino groups to each other. As the crosslinking agent, for example, aldehyde-based, carbodiimide-based, epoxide-based and imidazole-based crosslinking agents are preferred from the viewpoints of economy, safety and usability. Specific examples of the crosslinking agent include water soluble carbodiimides such as glutaraldehyde, 1-ethyl-3-(3-dimethylaminopropyl)carbodiimide-hydrochloride and 1-cyclohexyl-3-(2-morpholinyl-4-ethyl)carbodiimide-sulfonate.

Quantification of the degree of crosslinking can be appropriately determined depending on the type of the extracellular matrix component, the means of crosslinking, and the like. The degree of crosslinking may be 1% or more, preferably 2% or more, more preferably 4% or more, still more preferably 8% or more, and still even more preferably 12% or more, and may be 30% or less, preferably 20% or less, and more preferably 15% or less. The upper and lower limits of the degree of crosslinking can be combined as appropriate. For example, the degree of crosslinking may be 1% or more and 30% or less, preferably 2% or more and 20% or less, more preferably 4% or more and 15% or less, still more preferably 8% or more and 15% or less, and still even more preferably 12% or more and 15% or less. When the degree of crosslinking is within the above range, the extracellular matrix molecules can be appropriately dispersed, and have good redispersibility after dry storage.

When the amino groups in the extracellular matrix component are used for crosslinking, the degree of crosslinking can be quantified according to the TNBS method described in Glycobiology 2015, 25, 557, and the like. The degree of crosslinking by the TNBS method is preferably within the above range. The degree of crosslinking by the TNBS method is the proportion of amino groups used for crosslinking among the amino groups in the extracellular matrix.

The degree of crosslinking may be calculated by quantifying carboxyl groups. For example, an extracellular matrix component insoluble in water may be quantified by the TBO (toluidine blue O) method. The degree of crosslinking by the TBO method may be within the above range.

The content of the fragmented extracellular matrix component in the stromal cell-containing mixture may be 1 mass % or more, preferably 3 mass % or more, more preferably 10 mass % or more, still more preferably 20 mass % or more, still even more preferably 30 mass % or more, yet still even more preferably 40 mass % or more, yet still even more preferably 50 mass % or more, yet still even more preferably 60 mass % or more, yet still even more preferably 70 mass % or more, yet still even more preferably 80 mass % or more, yet still even more preferably 90 mass % or more, yet still even more preferably 95 mass % or more, and yet still even more preferably 98 mass % or more, or may be 99 mass % or less, preferably 95 mass % or less, and more preferably 90 mass % or less relative to the total amount of extracellular matrix-containing composition.

In process (a), the stromal cells, the cationic substance, the extracellular matrix component and the polyelectrolyte can be mixed in a suitable vessel such as a dish, tube, flask, bottle, well plate or cell culture insert. The mixing may be performed in a vessel used in process (b).

Further, in process (a), the stromal cells, the cationic substance and the fragmented extracellular matrix component can be mixed in a suitable vessel such as a dish, tube, flask, bottle, well plate or cell culture insert. The mixing may be performed in a vessel used in process (b).

Further, the stromal cell-containing mixture in process (a) may contain other components than the stromal cells, the cationic substance, the extracellular matrix component, the polyelectrolyte and the fragmented extracellular matrix component. Examples of the other components include gelling agents and cell culture media necessary for obtaining a first gel composition in process (b).

### Gelling Agent

Examples of the gelling agent include extracellular matrix components or fragmented extracellular matrix components; agarose; pectin; a combination of fibrinogen and thrombin. The gelling agent may be contained in advance in the stromal cell-containing mixture in process (a), or may be added, in process (b) described below, to the stromal cell-containing mixture obtained in process (a).

In process (b) subsequent to process (a), the stromal cell-containing mixture obtained in process (a) is gelled to obtain a first gel composition containing stromal cells. The method of gelling depends on the gelling agent used, and, for example, the stromal cell-containing mixture obtained in process (a) may be subjected to gelling conditions. Alternatively, a gelling agent may be added to the stromal cell-containing mixture obtained in process (a), and then the mixture may be subjected to gelling conditions.

For example, when an extracellular matrix component or a fragmented extracellular matrix component is used as the gelling agent, the gelling conditions include, for example, allowing the stromal cell-containing mixture obtained in process (a) to stand at about 37° C. As a result, the extracellular matrix component or the fragmented extracellular matrix component contained in the stromal cell-containing mixture in process (a) is gelled, whereby a first gel composition containing stromal cells is obtained. Alternatively, an extracellular matrix component or a fragmented extracellular matrix component may be further added to the stromal cell-containing mixture obtained in process (a), and the mixture may be gelled by allowing it to stand at about 37° C.

Further, when agarose is used as the gelling agent, agarose may be added to the stromal cell-containing mixture obtained in process (a), and, after dissolving the agarose under temperature conditions higher than or equal to the melting point of the agarose used, the mixture may be gelled by allowing it to stand under temperature conditions lower than or equal to the freezing point of the agarose used.

Further, when pectin is used as the gelling agent, pectin may be added to the stromal cell-containing mixture obtained in process (a). As a result, the pectin is gelled by divalent ions such as calcium ions contained in the stromal cell-containing mixture, whereby a first gel composition is obtained.

Further, fibrinogen and thrombin may be used as the gelling agent. Thrombin, a type of serine protease, cleaves the fibrinogen to form fibrin monomers. The fibrin monomers polymerize with each other by the action of calcium ions to form poorly-soluble fibrin polymers. The fibrin polymers are crosslinked in vivo by the action of factor XIII (fibrin stabilizing factor) to form mesh-like fibers called stabilized fibrin, causing blood coagulation. In this specification, a gel composition which is gelled due to fibrin polymers may also be referred to as a fibrin gel.

That is, process (b) of obtaining a first gel composition containing stromal cells may include a process of mixing thrombin and fibrinogen with the stromal cell-containing mixture obtained in process (a). The first gel composition containing stromal cells in process (b) may contain a fibrin gel as a first gel component.

In this case, process (b) of obtaining a first gel composition containing stromal cells preferably includes process (b1) of adding thrombin to the stromal cell-containing mixture obtained in process (a), and process (b2) of adding fibrinogen to the stromal cell-containing mixture to which thrombin has been added, whereby a fibrin gel is formed to gel the stromal cell-containing mixture.

When fibrinogen and thrombin are used as the gelling agent, the fibrinogen concentration in the stromal cell-containing mixture in process (b) is preferably 0.5 mg/mL or more and 25 mg/mL or less. The concentration of 0.5 mg/mL or more facilitates gelling when the fibrinogen is mixed with thrombin. The concentration of 25 mg/mL or less facilitates dissolving in the stromal cell-containing mixture. Further, thrombin is preferably dissolved or dispersed in the stromal cell-containing mixture in process (b).

If the stromal cell-containing mixture contains a substance with anticoagulant properties (typically, when heparin is selected as the polyelectrolyte), it is conceivable that fibrin may not be polymerized. For this reason, a substance with anticoagulant properties is not usually used together with fibrin. In the present embodiment, however, polymerization of fibrin is not inhibited, although it is not clear whether this is due to the combination of materials or the concentration being low.

In addition, the inventors have found that adding fibrinogen first to the stromal cell-containing mixture obtained in process (a) may cause the mixture to be gelled depending on the conditions. It is thought that the fibrinogen is cleaved by some protease contained in the stromal cell-containing mixture obtained in process (a), and forms fibrin monomers. Therefore, in mixing thrombin and fibrinogen with the stromal cell-containing mixture obtained in process (a), it is preferred to mix thrombin first and then mix fibrinogen with the mixture. Alternatively, a first gel composition containing stromal cells can be obtained by adding only fibrinogen as a gelling agent in process (b).

Thus, the first gel composition containing stromal cells formed in process (b) may contain a first gel component in which at least one of the extracellular matrix component, fragmented extracellular matrix component, agarose, pectin and fibrin monomers is gelled.

Then, in process (c), a target cell-containing mixture containing target cells, a cationic substance, an extracellular matrix component and a polyelectrolyte, or containing target cells, a cationic substance and a fragmented extracellular matrix component is obtained. That is, in process (c), a target cell-containing mixture containing target cells, a cationic substance, an extracellular matrix component and a polyelectrolyte is obtained, or a target cell-containing mixture containing target cells, a cationic substance and a fragmented extracellular matrix component is obtained.

### Target Cells

The term “target cells” as used herein refers to cells whose maintenance is to be controlled using stromal cells. The target cells are not particularly limited, and cells derived from mammals such as humans, monkeys, dogs, cats, rabbits, pigs, cows, mice and rats can be used. Also, the site of origin of the target cells is not particularly limited, and the target cells may be somatic cells, such as those derived from bone, muscle, viscera, nerve, brain, skin and blood, or may be reproductive cells, or cancer cells.

Examples of the somatic cells derived from blood include immune cells such as lymphocytes, neutrophils, macrophages and dendritic cells. Examples of the cancer cells include the cells of gastric cancer, esophageal cancer, colorectal cancer, colon cancer, rectal cancer, pancreatic cancer, breast cancer, ovarian cancer, prostate cancer, renal cell cancer and liver cancer.

Moreover, the target cells may be pluripotent stem cells such as induced pluripotent stem cells (iPS cells) and embryonic stem cells (ES cells) or may be tissue stem cells.

The target cells may be cultured cells such as primary cells, subcultured cells and cell line cells. These target cells may be used singly or in combination of two or more.

The cationic substance, the extracellular matrix component, the polyelectrolyte and the fragmented extracellular matrix component used in process (c) may be those used in process (a).

In process (c), the target cells, the cationic substance, the extracellular matrix component and the polyelectrolyte can be mixed in a suitable vessel such as a dish, tube, flask, bottle, well plate or cell culture insert. The mixing may be performed in a vessel used in process (d).

Further, in process (c), the target cells, the cationic substance and the fragmented extracellular matrix component can be mixed in a suitable vessel such as a dish, tube, flask, bottle, well plate or cell culture insert. The mixing may be performed in a vessel used in process (d).

Further, the target cell-containing mixture in process (c) may contain other components than the target cells, the cationic substance, the extracellular matrix component, the polyelectrolyte and the fragmented extracellular matrix component. Examples of the other components include gelling agents and cell culture media necessary for obtaining a second gel composition containing target cells in process (e).

Examples of the gelling agent include extracellular matrix components or fragmented extracellular matrix components; agarose; pectin; a combination of fibrinogen and thrombin. The gelling agent may be contained in advance in the target cell-containing mixture in process (c), or may be added, in process (e) described below, to the target cell-containing mixture obtained in process (c).

Subsequently, in process (d), the target cell-containing mixture is placed in contact with the first gel composition containing stromal cells. The method of placement is not particularly limited as long as the target cells are in contact with the first gel composition containing stromal cells, and the target cells may be placed on the first gel composition containing stromal cells or may be placed in the first gel composition containing stromal cells.

When placing the target cells in the first gel composition containing stromal cells, it is preferred that, after process (e), which will be described later, the stromal cell-containing mixture obtained in process (a) is laminated on the second gel composition containing target cells, and the stromal cell-containing mixture laminated on the second gel composition containing target cells is gelled in the same manner as in process (b), whereby a third gel composition containing stromal cells is laminated on the second gel composition containing target cells. That is, the second gel composition containing target cells is placed in contact with the first gel composition containing stromal cells. Then, the stromal cell-containing mixture is laminated on the second gel composition and gelled. As a result, the third gel composition containing stromal cells is laminated on the second gel composition.

After process (d), the target cell-containing mixture obtained in process (c) is gelled in process (e) to obtain the second gel composition containing target cells. The method of gelling depends on the gelling agent used, and, for example, the target cell-containing mixture obtained in process (c) may be subjected to gelling conditions. Alternatively, a gelling agent may be added to the target cell-containing mixture obtained in process (c), and then the mixture may be subjected to gelling conditions.

The gelling conditions for obtaining the second gel composition containing target cells in process (d) are the same as the gelling conditions for obtaining the first gel composition containing stromal cells in process (b) except that the target cells are used instead of the stromal cells.

In process (d), due to the target cell-containing mixture being gelled, the target cell-containing mixture placed in contact with the first gel composition containing stromal cells is prevented from migrating or spreading from the position of the first gel composition containing stromal cells. Therefore, the target cells can be maintained at predetermined positions in a three-dimensional cellular tissue.

Subsequently, in process (f), the first gel composition containing stromal cells obtained in process (b) and the second gel composition containing target cells obtained in process (e) are incubated to obtain a three-dimensional cellular tissue. The period of time for culturing the first gel composition containing stromal cells and the second gel composition containing target cells to obtain a three-dimensional cellular tissue may be 5 minutes to 72 hours. Process (f) promotes adhesion between stromal cells contained in the first gel composition and between target cells contained in the second gel composition, whereby the effect of stabilizing the three-dimensional cellular tissue is achieved. In addition, since the target cells are prevented from migrating or spreading from position of the first gel composition containing stromal cells, the effect of maintaining the target cells at predetermined positions in the three-dimensional cellular tissue is achieved. This makes it possible to construct an organ that requires more structural three-dimensional control. In addition, since the thickness of the target cells can be maintained, a large tissue can be cultured for a long period of time. Furthermore, due to the target cells being maintained at predetermined positions in the three-dimensional cellular tissue, it is possible to easily evaluate the size, degree of migration, and the like of the target cells, and thus easily evaluate, for example, sensitivity to anticancer drugs, metastasis of cancer cells, degree of invasion of cancer cells, and the like, whereby screening for anticancer drugs, and the like, can be easily performed.

In process (f), culture of stromal cells and target cells can be performed under culture conditions suitable for the cells to be cultured. Those skilled in the art can select an appropriate medium according to the types of stromal cells and target cells and desired functions. Examples of the medium include, but are not limited to, media such as DMEM, EMEM, MEMα, RPMI-1640, McCoy's 5A and Ham's F-12, and media obtained by adding about 1 vol % to 20 vol % of serum to these media. Examples of serum include bovine serum (CS), fetal bovine serum (FBS), fetal horse serum (HBS), and the like. Conditions such as the temperature and atmospheric composition of the culture environment may also be adjusted according to the stromal cells and target cells to be cultured.

The vessel used in process (f) may be the same as the vessel used in process (b). In process (f), the vessel used in process (b) may be used as it is, or another vessel may be used.

Upon culture of stromal cells and target cells, a substance for suppressing deformation of the obtained three-dimensional cellular tissue (such as tissue contraction, peeling at the tissue edge, or the like) may be added to the medium. Examples of such a substance include, but are not limited to, Y-27632, which is a Rho-associated coiled-coil forming kinase/Rho binding kinase (ROCK) inhibitor.

Process (f) may be performed after processes (a) to (e) are performed two or more times. By repeating processes (a) to (e), a three-dimensional cellular tissue in which the target cells are placed at different positions on and in the stromal cellular tissue. That is, a three-dimensional cellular tissue having an increased thickness can be produced.

Further, processes (a) to (e) may be repeated using a different target cell population for each repetition to form a three-dimensional cellular tissue composed of different types of target cells.

### Cell Aggregate

The production method of the present embodiment may further include: before process (b) of obtaining a first gel composition containing stromal cells, process (a′) of applying an external force to the stromal cell-containing mixture obtained in process (a) to obtain a stromal cell aggregate containing stromal cells, a cationic substance, an extracellular matrix component and a polyelectrolyte, or a stromal cell aggregate containing stromal cells, a cationic substance and a fragmented extracellular matrix component; and, in process (b) of obtaining a first gel composition containing stromal cells, a process of gelling, as the stromal cell-containing mixture obtained in process (a), the stromal cell aggregate obtained in process (a′) to obtain a first gel composition containing stromal cells.

In this case, the method of producing three-dimensional cellular tissues according to the present embodiment includes: process (a) of obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; process (a′) of applying an external force to the stromal cell-containing mixture to obtain a stromal cell aggregate containing stromal cells, a cationic substance, an extracellular matrix component and a polyelectrolyte, or a stromal cell aggregate containing stromal cells, a cationic substance and a fragmented extracellular matrix component; process (b′) of gelling the stromal cell aggregate to obtain a first gel composition containing stromal cells; process (c) of obtaining a target cell-containing mixture containing target cells, a cationic substance, an extracellular matrix component and a polyelectrolyte, or a target cell-containing mixture containing target cells, a cationic substance and a fragmented extracellular matrix component; process (d) of placing the target cell-containing mixture in contact with the first gel composition; process (e) of gelling the target cell-containing mixture to obtain a second gel composition containing target cells; and process (f) of incubating the first gel composition and the second gel composition to obtain a three-dimensional cellular tissue.

Furthermore, the production method of the present embodiment may further include: before process (d) of placing the target cell-containing mixture in contact with the first gel composition, process (c′) of applying an external force to the target cell-containing mixture obtained in process (c) to obtain a target cell aggregate containing target cells, a cationic substance, an extracellular matrix component and a polyelectrolyte, or a target cell aggregate containing target cells, a cationic substance and a fragmented extracellular matrix component; in process (d) of placing the target cell-containing mixture in contact with the first gel composition, a process of placing, as the target cell-containing mixture, the target cell aggregate obtained in process (c′) in contact with the first gel composition; and, in process (e) of obtaining a second gel composition containing target cells, a process of gelling, as the target cell-containing mixture obtained in process (c), the target cell aggregate obtained in process (c′) to obtain a second gel composition containing target cells.

In this case, the method of producing three-dimensional cellular tissues according to the present embodiment includes: process (a) of obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; process (b) of gelling the stromal cell-containing mixture to obtain a first gel composition containing stromal cells; process (c) of obtaining a target cell-containing mixture containing target cells, a cationic substance, an extracellular matrix component and a polyelectrolyte, or a target cell-containing mixture containing target cells, a cationic substance and a fragmented extracellular matrix component; process (c′) of applying an external force to the target cell-containing mixture to obtain a target cell aggregate containing target cells, a cationic substance, an extracellular matrix component and a polyelectrolyte, or a target cell aggregate containing target cells, a cationic substance and a fragmented extracellular matrix component; process (d′) of placing the target cell aggregate in contact with the first gel composition; process (e′) of gelling the target cell aggregate to obtain a second gel composition containing target cells; and process (f) of incubating the first gel composition and the second gel composition to obtain a three-dimensional cellular tissue.

Furthermore, the production method of the present embodiment may further include: before process (b) of obtaining a first gel composition containing stromal cells, process (a′) of applying an external force to the stromal cell-containing mixture obtained in process (a) to obtain a stromal cell aggregate containing stromal cells, a cationic substance, an extracellular matrix component and a polyelectrolyte, or a stromal cell aggregate containing stromal cells, a cationic substance and a fragmented extracellular matrix component; in process (b) of obtaining a first gel composition containing stromal cells, a process of gelling, as the stromal cell-containing mixture obtained in process (a), the stromal cell aggregate obtained in process (a′) to obtain a first gel composition containing stromal cells; before process (d) of placing the target cell-containing mixture in contact with the first gel composition, process (c′) of applying an external force to the target cell-containing mixture obtained in process (c) to obtain a target cell aggregate containing target cells, a cationic substance, an extracellular matrix component and a polyelectrolyte, or a target cell aggregate containing target cells, a cationic substance and a fragmented extracellular matrix component; in process (d) of placing the target cell-containing mixture in contact with the first gel composition, a process of placing, as the target cell-containing mixture, the target cell aggregate obtained in process (c′) in contact with the first gel composition; and, in process (e) of obtaining a second gel composition containing target cells, a process of gelling, as the target cell-containing mixture obtained in process (c), the target cell aggregate obtained in process (c′) to obtain a second gel composition containing target cells.

In this case, the method of producing three-dimensional cellular tissues according to the present embodiment includes: process (a) of obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; process (a′) of obtaining a stromal cell aggregate containing stromal cells, a cationic substance, an extracellular matrix component and a polyelectrolyte, or a stromal cell aggregate containing stromal cells, a cationic substance and a fragmented extracellular matrix component; process (b′) of gelling the stromal cell aggregate to obtain a first gel composition containing stromal cells; process (c) of obtaining a target cell-containing mixture containing target cells, a cationic substance, an extracellular matrix component and a polyelectrolyte, or a target cell-containing mixture containing target cells, a cationic substance and a fragmented extracellular matrix component; process (c′) of applying an external force to the target cell-containing mixture to obtain a target cell aggregate containing target cells, a cationic substance, an extracellular matrix component and a polyelectrolyte, or a target cell aggregate containing target cells, a cationic substance and a fragmented extracellular matrix component; process (d′) of placing the target cell aggregate in contact with the first gel composition; process (e′) of gelling the target cell aggregate to obtain a second gel composition containing target cells; and process (f) of incubating the first gel composition and the second gel composition to obtain a three-dimensional cellular tissue.

The term “cell aggregate” as used herein refers to a structure composed of cells combined into a single unit. The cell aggregate includes cell precipitates obtained by centrifugation, filtration, or the like. In an embodiment, the cell aggregate is a slurry-like viscous body. The term “slurry-like viscous body” refers to a gel-like cell aggregate as described in Akihiro Nishiguchi et al., Cell-cell crosslinking by bio-molecular recognition of heparin-based layer-by-layer nanofilms, Macromol Biosci., 15 (3), 312-317, 2015.

The cell aggregate may be formed by allowing the stromal cell-containing mixture obtained in process (a) or the target cell-containing mixture obtained in process (c) to stand in an appropriate vessel. Further, the cell aggregate may be formed by placing the stromal cell-containing mixture obtained in process (a) or the target cell-containing mixture obtained in process (c) in an appropriate vessel, and subjecting it to, for example, centrifugation, magnetic separation, filtration, or the like to aggregate the cells. That is, applying an external force in process (a′) or process (c′) may refer to allowing the mixture to stand to apply gravity, or performing centrifugation, magnetic separation, filtration, or the like. After the cells are aggregated by standing, centrifugation, magnetic separation, filtration, or the like, the liquid part may be removed or may not be removed.

The vessel used in process (a′) or process (c′) may be a culture vessel used for cell culture. The culture vessel may be a vessel having a material and a shape typically used for cell or microorganism culture. Examples of the material of the culture vessel include, but are not limited to, glass, stainless steel, plastic, and the like. Examples of the culture vessel include, but are not limited to, a dish, tube, flask, bottle, well plate, cell culture insert, and the like. Preferably, the vessel is, at least in part, made of a material that allows liquid to pass through without allowing cells in the liquid to pass through. Examples of such a vessel include, but are not limited to, cell culture inserts, such as a Transwell (registered trademark) insert, Netwell (registered trademark) insert, Falcon (registered trademark) cell culture insert, and Millicell (registered trademark) cell culture insert.

The conditions of centrifugation are not particularly limited as long as they do not adversely affect the growth of stromal cells and target cells. For example, the stromal cells or target cells can be aggregated to obtain a stromal cell aggregate or target cell aggregate by placing the stromal cell-containing mixture or target cell-containing mixture in a cell culture insert and centrifuging it at 10° C. at 400×g for 1 minute.

Process (b′) of gelling the stromal cell aggregate to obtain a first gel composition containing stromal cells can be performed in the same manner as in process (b) of gelling the stromal cell-containing mixture obtained in process (a) described above to obtain a first gel composition.

Further, process (c′) of gelling the target cell aggregate to obtain a second gel composition containing target cells can be performed in the same manner as in process (c) of gelling the target cell-containing mixture obtained in process (c) described above to obtain a second gel composition containing target cells.

For example, when an extracellular matrix component is used as the gelling agent, the stromal cell aggregate obtained in process (a′) or the target cell aggregate obtained in process (c′) may be allowed to stand at about 37ºC, or an extracellular matrix component may be added to the stromal cell aggregate obtained in process (a′) or the target cell aggregate obtained in process (c′) and then the cell aggregate may be gelled by allowing it to stand at about 37° C.

Further, when agarose is used as the gelling agent, agarose may be added to the stromal cell aggregate obtained in process (a′) or the target cell aggregate obtained in process (c′), and, after dissolving the agarose under temperature conditions higher than or equal to the melting point of the agarose used, the cell aggregate may be gelled by allowing it to stand under temperature conditions lower than or equal to the freezing point of the agarose used.

Further, when pectin is used as the gelling agent, pectin may be added to the stromal cell aggregate obtained in process (a′) or the target cell aggregate obtained in process (c′). As a result, the pectin is gelled by divalent ions such as calcium ions contained in the stromal cell aggregate or the target cell aggregate, whereby a first gel composition containing stromal cells or a second gel composition containing target cells is obtained.

Further, fibrinogen and thrombin may be used as the gelling agent. That is, process (b′) of obtaining a first gel composition containing stromal cells may include a process of mixing thrombin and fibrinogen with the stromal cell aggregate obtained in process (a′). The first gel composition containing stromal cells in process (b′) may contain a fibrin gel as a first gel component.

In this case, process (b′) of obtaining a first gel composition containing stromal cells preferably include process (b1′) of adding thrombin to the stromal cell aggregate obtained in process (a′), and process (b2′) of adding fibrinogen to the stromal cell aggregate to which thrombin has been added, whereby a fibrin gel is formed to gel the stromal cell aggregate.

Further, process (e′) of obtaining a second gel composition containing target cells may include a process of mixing thrombin and fibrinogen with the target cell aggregate obtained in process (c′). The second gel composition containing target cells in process (e′) may contain a fibrin gel as a second gel component.

In this case, process (e′) of obtaining a second gel composition containing target cells preferably include process (e1′) of adding thrombin to the target cell aggregate obtained in process (c′) and process (e2′) of adding fibrinogen to the target cell aggregate to which thrombin has been added, whereby a fibrin gel is formed to gel the target cell aggregate.

Subsequently, in process (f), the first gel composition containing stromal cells obtained in process (b′) and the second gel composition containing target cells obtained in process (e′) are incubated to obtain a three-dimensional cellular tissue. Process (f) is the same as that described above.

### Three-Dimensional Cellular Tissue

One embodiment of the present invention provides a three-dimensional cellular tissue in which a target cell gel section containing target cells, a cationic substance, an extracellular matrix component, a polyelectrolyte and a second gel component or a target cell gel section containing target cells, a cationic substance, a fragmented extracellular matrix component and a second gel component is placed in contact with a stromal cell gel section containing stromal cells, a cationic substance, an extracellular matrix component, a polyelectrolyte and a first gel component or a stromal cell gel section containing stromal cells, a cationic substance, a fragmented extracellular matrix component and a first gel component. In the three-dimensional cellular tissue of the present embodiment, the target cells are placed at predetermined positions on and in the stromal cellular tissue.

The three-dimensional cellular tissue of the present embodiment may be a three-dimensional cellular tissue in which a target cell gel section containing target cells, a cationic substance, an extracellular matrix component, a polyelectrolyte and a second gel component is placed in contact with a stromal cell gel section containing stromal cells, a cationic substance, an extracellular matrix component, a polyelectrolyte and a first gel component. The three-dimensional cellular tissue of the present embodiment may be a three-dimensional cellular tissue in which a target cell gel section containing target cells, a cationic substance, a fragmented extracellular matrix component and a second gel component is placed in contact with a stromal cell gel section containing stromal cells, a cationic substance, an extracellular matrix component, a polyelectrolyte and a first gel component.

Furthermore, the three-dimensional cellular tissue of the present embodiment may be a three-dimensional cellular tissue in which a target cell gel section containing target cells, a cationic substance, an extracellular matrix component, a polyelectrolyte and a second gel component is placed in contact with a stromal cell gel section containing stromal cells, a cationic substance, a fragmented extracellular matrix component and a first gel component. The three-dimensional cellular tissue of the present embodiment may be a three-dimensional cellular tissue in which a target cell gel section containing target cells, a cationic substance, a fragmented extracellular matrix component and a second gel component is placed in contact with a stromal cell gel section containing stromal cells, a cationic substance, a fragmented extracellular matrix component and a first gel component.

In the three-dimensional cellular tissue of the present embodiment, the state in which the target cell gel section is placed in contact with the stromal cell gel section refers to a state in which the target cell gel section is placed on the stromal cell gel section or a state in which the target cell gel section is placed in the stromal cell gel section. The state in which the stromal cell gel section and the target cell gel section are in contact with each other may be a state in which the target cell gel section is sandwiched between two stromal cell gel sections.

In the three-dimensional cellular tissue of the present embodiment, the stromal cells, the target cells, the cationic substance, the extracellular matrix component, the polyelectrolyte, the first and second gel components and the fragmented extracellular matrix component are the same as those described above.

### EXAMPLES

The present invention will be described in more detail using the examples, but the present invention is not limited to the following examples.

### Experimental Example 1

### Production of Three-Dimensional Cellular Tissues

Three-dimensional cellular tissues in which cancer cells containing fibrin gel were laminated on stromal cells containing fibrin gel, and three-dimensional cellular tissues in which cancer cells containing no fibrin gel are laminated on stromal cells containing no fibrin gel were produced. The cancer cells were target cells. Neonatal human dermal fibroblasts NHDF (product number “CC-2509”, Lonza) and human umbilical vein endothelial cells GFP-HUVEC (product number “cAP-0001GFP”, Funakoshi Co., Ltd.) were used as the stromal cells, and human alveolar basal epithelial adenocarcinoma cells A549 (ATCC CCL-185) were used as the cancer cells.

### Production of Stromal Cells Containing Fibrin Gel

A DMEM medium (product number “043-30085”, FUJIFILM Wako Pure Chemical Corporation) containing 10% fetal bovine serum (FBS) and 1% antibiotic solution (penicillin, streptomycin) was prepared (hereinafter, also referred to as a “general-purpose culture medium”). Further, a medium was prepared by mixing a general-purpose culture medium and a vascular cell growth medium (product name “EGM-2MV”, product number “CC-3202”, Lonza) at 1:1 (volume ratio) (hereinafter, also referred to as a “dedicated culture medium”).

A thrombin solution was prepared by dissolving thrombin (product number “T4648-10KU”, Sigma) in the general-purpose culture medium described above to produce final concentration of 10 Unit/mL.

A fibrinogen solution was prepared by dissolving fibrinogen (product number “F8630-5G”, Sigma) in the DMEM medium to produce final concentration of 10 mg/mL. Further, a medium prepared by mixing a fibrinogen solution and a general-purpose culture medium at 1:1 (volume ratio) (hereinafter, also referred to as a “Hep/Col fibrinogen-added medium”) and a medium prepared by mixing a fibrinogen solution and a general-purpose culture medium containing 0.05 mg/mL collagen microfiber (CMF; product number “307-31611”, NH Foods Ltd.) at 1:1 (volume ratio) (hereinafter, also referred to as a “CMF fibrinogen-added medium”) were prepared. The fibrinogen was a gelling agent.

Then, 2×10⁶ NHDF cells and 3×10⁴ GFP-HUVEC cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma). The heparin was a polyelectrolyte, and the collagen was an extracellular matrix component.

Then, the obtained cell suspension was centrifuged at room temperature at 1,000×g for 1 minute, and, after removing the supernatant, resuspended in the thrombin solution. Subsequently, the thrombin solution in which the cells were suspended and the Hep/Col fibrinogen-added medium or the CMF fibrinogen-added medium were mixed at 1:2 (volume ratio), and 50 μL of the mixed solution was seeded in a 24-well cell culture insert (product number “3470”, Corning Incorporated).

Then, the cell culture insert was allowed to stand in a CO₂ incubator (37° C., 5% CO₂) until the mixture was gelled to thereby obtain a cell culture insert containing fibrin gel.

### Production of Stromal Cells Containing No Fibrin Gel

2×10⁶ NHDF cells and 3×10⁴ GFP-HUVEC cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma).

Then, the obtained cell suspension was centrifuged at room temperature at 1,000×g (gravitational acceleration) for 1 minute, and, after removing the supernatant, resuspended in an appropriate amount of the dedicated culture medium. Subsequently, 50 μL of the cell suspension was seeded in a 24-well cell culture insert (product number “3470”, Corning Incorporated). Then, the cell culture insert was allowed to stand in a CO₂ incubator (37° C., 5% CO₂) for 24 hours to thereby obtain a cell culture insert containing no fibrin gel.

### Lamination of Cancer Cells Containing Fibrin Gel

1×10⁵ A549 cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma).

Then, the obtained cell suspension was centrifuged at room temperature at 1,000×g for 1 minute, and, after removing the supernatant, resuspended in the thrombin solution. Subsequently, the thrombin solution in which the cells were suspended and the Hep/Col fibrinogen-added medium or the CMF fibrinogen-added medium were mixed at 1:2 (volume ratio), and 5 μL of the mixed solution was seeded in the center part of the cell culture insert containing fibrin gel or cell culture insert containing no fibrin gel obtained as above.

Then, an appropriate amount of the dedicated culture medium was added to the cell culture insert containing fibrin gel or cell culture insert containing no fibrin gel described above and incubated in a CO₂ incubator (37° C., 5% CO₂) for 7 days. During the culture, the medium was exchanged as appropriate.

### Lamination of Cancer Cells Containing No Fibrin Gel

1×10⁵ A549 cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma).

Then, the cell suspension was centrifuged at room temperature at 1,000×g (gravitational acceleration) for 1 minute, and, after removing the supernatant, resuspended in an appropriate amount of the dedicated culture medium. Subsequently, 5 μL of the cell suspension was seeded in the center part of the cell culture insert containing fibrin gel or cell culture insert containing no fibrin gel obtained as above.

Then, an appropriate amount of the dedicated culture medium was added to the cell culture insert containing fibrin gel or cell culture insert containing no fibrin gel described above and incubated in a CO₂ incubator (37° ° C., 5% CO₂) for 7 days.

### Evaluation of Positions of Cancer Cells

On day 8 of culture, observation was performed with a microscope system (Operetta CLS, PerkinElmer) in a live cell fluorescence mode (37° ° C., 5% CO₂, fluorescence: Alexa 594). When the stained regions derived from the cancer cells were within the range of liquid droplets (fibrin gel) immediately after seeding, it was evaluated as “good”, and when the stained regions have spread or migrated outside the range, it was evaluated as “poor”. The results are shown in Table 1. In the table, “Hep/Col” indicates the case where gelation was performed using the Hep/Col fibrinogen-added medium, and “CMF” indicates the case where gelation was performed using the CMF fibrin gel-added medium.

TABLE 1

| | | | Cancer cells | | |
|---|---|---|---|---|---|
| | | | | | Containing |
| | | | Containing fibrin gel | | no fibrin |
| | | | Hep/Col | CMF | gel |
| Stromal | Containing | Hep/Col | Good | Good | Poor |
| cells | fibrin gel | CMF | Good | Good | Poor |

| | | | |
|---|---|---|---|
| Containing no fibrin gel | Poor | Poor | Poor |

As shown in Table 1, when both the cancer cells and stromal cells contained fibrin gel, the positional evaluation of the cancer cells in the three-dimensional cellular tissue was “good” regardless of the heparin and collagen-containing fibrin gel or CMF-containing fibrin gel, indicating that the cancer cells have not migrated on the stromal cellular tissue. On the other hand, when either the cancer cells or the stromal cells did not contain fibrin gel, the positional evaluation of the cancer cells in the three-dimensional cellular tissue was “poor”, indicating that the cancer cells have spread or migrated on the stromal cellular tissue.

### Experimental Example 2

Production of Three-Dimensional Cellular Tissue with Cancer Cells Containing Fibrin Gel Placed on Stromal Cells Containing Fibrin Gel

Three-dimensional cellular tissues in which cancer cells containing fibrin gel were laminated on stromal cells containing fibrin gel were produced. Neonatal human dermal fibroblasts NHDF (product number “CC-2509”, Lonza) and human umbilical vein endothelial cells GFP-HUVEC (product number “cAP-0001GFP”, Funakoshi Co., Ltd.) were used as the stromal cells, and human alveolar basal epithelial adenocarcinoma cells A549 (ATCC CCL-185) were used as the cancer cells.

### Production of Stromal Cells Containing Fibrin Gel

A thrombin solution was prepared by dissolving thrombin (product number “T4648-10KU”, Sigma) in a general-purpose culture medium to produce final concentration of 10 Unit/mL.

Then, 2×10⁶ NHDF cells and 3×10⁴ GFP-HUVEC cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma). Then, the obtained cell suspension was centrifuged at room temperature at 1,000×g for 1 minute, and, after removing the supernatant, resuspended in the thrombin solution. Subsequently, the thrombin solution in which the cells were suspended and the Hep/Col fibrinogen-added medium were mixed at 1:2 (volume ratio), and 50 μL of the mixed solution was seeded in a 24-well cell culture insert (product number “3470”, Corning Incorporated).

Then, the cell culture insert was allowed to stand in a CO₂ incubator (37° C., 5% CO₂) until the mixture was gelled to thereby obtain a cell culture insert containing fibrin gel.

### Lamination of Cancer Cells Containing Fibrin Gel

1×10⁵ A549 cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma).

Then, the obtained cell suspension was centrifuged at room temperature at 1,000×g for 1 minute, and, after removing the supernatant, resuspended in the thrombin solution. Subsequently, the thrombin solution in which the cells were suspended and the CMF fibrinogen-added medium were mixed at 1:2 (volume ratio), and 5 μL of the mixed solution was seeded at two positions in the center part of the cell culture insert containing fibrin gel obtained as above.

Then, an appropriate amount of the dedicated culture medium was added to the cell culture insert containing fibrin gel described above and incubated in a CO₂ incubator (37° C., 5% CO₂) for 8 days. During the culture, the medium was exchanged as appropriate.

### Fluorescence Observation

On day 8 of culture, observation was performed with a microscope system (Operetta CLS, PerkinElmer) in a live cell fluorescence mode (37° C., 5% CO₂, GFP detection wavelength Ex 494 nm/Em 526 nm, TMR detection wavelength Ex 555 nm/Em 585 nm).

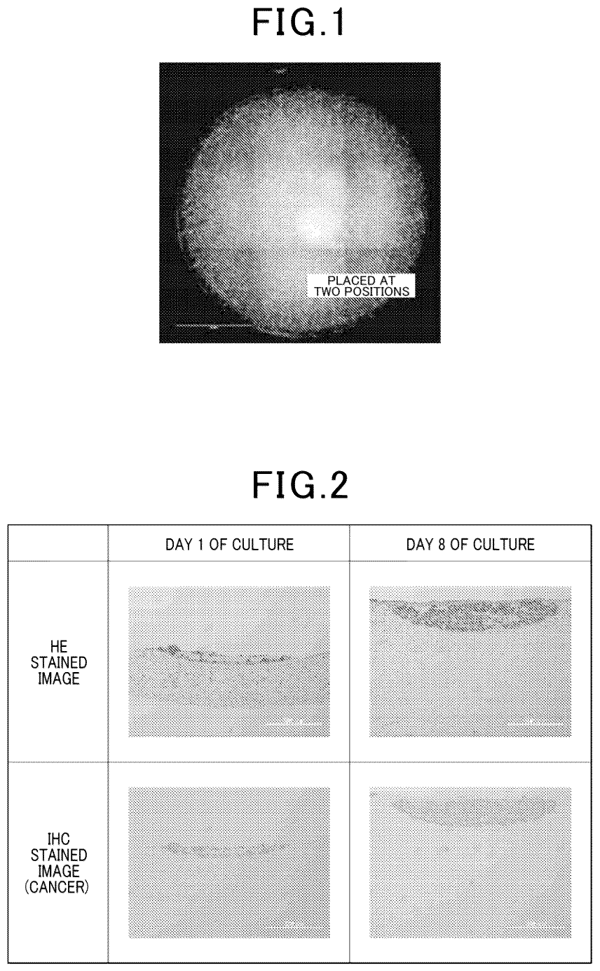

FIG. 1 is a representative micrograph showing the positions of cancer cells in a three-dimensional cellular tissue obtained as above. As shown in FIG. 1, the stained regions derived from the cancer cells were found to be within the range of liquid droplets (fibrin gel) immediately after seeding, and not to have migrated or spread on the stromal cellular tissue.

### Tissue Section Analysis

On day 1 and day 8 of culture of cell aggregate, the three-dimensional cellular tissues were fixed using a formalin buffer solution (product number “062-01661”, FUJIFILM Wako Pure Chemical Corporation). Then, each three-dimensional cellular tissue was removed from the cell culture insert, embedded in paraffin, and cut along the line passing through the center of gravity in a direction perpendicular to the upper surface of the three-dimensional cellular tissue (upper surface of the cell culture insert) to prepare thin slices. Subsequently, the thin slices were subjected to hematoxylin-eosin (HE) staining and immunochemistry staining (IHC staining) using an anti-human EpCAM antibody (product number “36746S”, Cell Signaling Technology), and the positions of cancer cells in the HE stained and IHC stained three-dimensional cellular tissues were observed.

FIG. 2 is representative micrographs of three-dimensional cellular tissue fragments on day 1 and day 8 of culture. As shown in FIG. 2, the cancer cells on day 8 of culture were found not to have migrated from the positions on day 1 of culture on the stromal cells.

### Experimental Example 3

(Production of Three-Dimensional Cellular Tissue with Cancer Cells Containing No Fibrin Gel Placed on Stromal Cells Containing Fibrin Gel)

Three-dimensional cellular tissues in which cancer cells containing no fibrin gel were laminated on stromal cells containing fibrin gel were produced. Neonatal human dermal fibroblasts NHDF (product number “CC-2509”, Lonza) and human umbilical vein endothelial cells RFP-HUVEC (product number “cAP-0001 RFP”, Funakoshi Co., Ltd.) were used as the stromal cells, and GFP-expressing human alveolar basal epithelial adenocarcinoma cells A549 (“AKR-209”, CELL BIOLABS, Inc.) were used as the cancer cells.

### Production of Stromal Cells Containing Fibrin Gel

A thrombin solution was prepared by dissolving thrombin (product number “T4648-10KU”, Sigma) in a general-purpose culture medium to produce final concentration of 10 Unit/mL.

Then, 2×10⁶ NHDF cells and 3×10⁴ RFP-HUVEC cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma). Then, the obtained cell suspension was centrifuged at room temperature at 1,000×g for 1 minute, and, after removing the supernatant, resuspended in the thrombin solution. Subsequently, the thrombin solution in which the cells were suspended and the Hep/Col fibrinogen-added medium were mixed at 1:2 (volume ratio), and 50 μL of the mixed solution was seeded in a 24-well cell culture insert (product number “3470”, Corning Incorporated).

Then, the cell culture insert was allowed to stand in a CO₂ incubator (37° C., 5% CO₂) until the mixture was gelled to thereby obtain a cell culture insert containing fibrin gel.

### Lamination of Cancer Cells Containing No Fibrin Gel

1×10⁵ GFP-expressing A549 cells were suspended in a solution obtained by diluting a general-purpose culture medium containing 0.05 mg/mL collagen microfiber (CMF) to ⅓ with a general-purpose culture medium.

Subsequently, 5 μL of the cell suspension was seeded in the center part of the cell culture insert containing fibrin gel obtained as above.

Then, an appropriate amount of the dedicated culture medium was added to the cell culture insert containing fibrin gel described above and incubated in a CO₂ incubator (37° ° C., 5% CO₂) for 8 days.

### Fluorescence Observation

On day 8 of culture, observation was performed with a microscope system (Operetta CLS, PerkinElmer) in a live cell fluorescence mode (37° C., 5% CO₂, GFP detection wavelength Ex 494 nm/Em 526 nm, RFP detection wavelength Ex 555 nm/Em 583 nm).

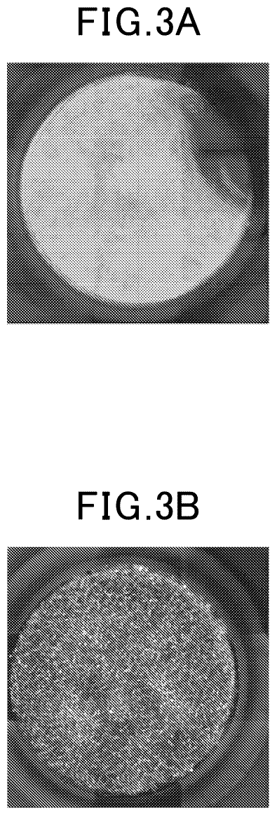

FIG. 3A is a representative micrograph of a three-dimensional cellular tissue on day 8 of culture, in which cancer cells are stained. FIG. 3B is a representative micrograph of a three-dimensional cellular tissue on day 8 of culture, in which stromal cells are stained. As shown in FIG. 3A, the stained regions derived from the cancer cells were found not to be within the range of liquid droplets (fibrin gel) immediately after seeding onto the stromal cells but have been spread over the entire surface.

### Experimental Example 4

Production of Three-Dimensional Cellular Tissue with Cancer Cells Containing Fibrin Gel

### Placed in Stromal Cells Containing Fibrin Gel

Three-dimensional cellular tissues in which cancer cells containing fibrin gel were placed in stromal cells containing fibrin gel were produced. Neonatal human dermal fibroblasts NHDF (product number “CC-2509”, Lonza) and human umbilical vein endothelial cells GFP-HUVEC (product number “cAP-0001GFP”, Funakoshi Co., Ltd.) were used as the stromal cells, and human colon adenocarcinoma cells HT29 (ATCC HTB-38) were used as the cancer cells.

### Production of Stromal Cells Containing Fibrin Gel

A thrombin solution was prepared by dissolving thrombin (product number “T4648-10KU”, Sigma) in a general-purpose culture medium to produce final concentration of 10 Unit/mL.

Then, 2×10⁶ NHDF cells and 3×10⁴ GFP-HUVEC cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma). Then, the obtained cell suspension was centrifuged at room temperature at 1,000×g for 1 minute, and, after removing the supernatant, resuspended in the thrombin solution. Subsequently, the thrombin solution in which the cells were suspended and the Hep/Col fibrinogen-added medium were mixed at 1:2 (volume ratio), and 50 μL of the mixed solution was seeded in a 24-well cell culture insert (product number “3470”, Corning Incorporated).

Then, the cell culture insert was allowed to stand in a CO₂ incubator (37° C., 5% CO₂) until the mixture was gelled to thereby obtain a cell culture insert containing fibrin gel.

Lamination of Cancer Cells Containing Fibrin Gel 1×10⁵ HT29 cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma).

Then, the obtained cell suspension was centrifuged at room temperature at 1,000×g for 1 minute, and, after removing the supernatant, resuspended in the thrombin solution. Subsequently, the thrombin solution in which the cells were suspended and the CMF fibrinogen-added medium were mixed at 1:2 (volume ratio), and 5 μL of the mixed solution was seeded in the center part of the cell culture insert containing fibrin gel obtained as above.

Then, the cell culture insert was allowed to stand in a CO₂ incubator (37° C., 5% CO₂) until the mixture was gelled to thereby obtain a cell culture insert containing fibrin gel on which the cancer cells were laminated.

### Lamination of Stromal Cells Containing Fibrin Gel

2×10⁶ NHDF cells and 3×10⁴ GFP-HUVEC cells were suspended in a 50 mM tris-HCl buffer solution (pH 7.4) containing 0.05 mg/mL heparin (product number “H3149-100KU”, Sigma) and 0.05 mg/mL collagen (product number “ASC-1-100-100”, Sigma).

Then, the obtained cell suspension was centrifuged at room temperature at 1,000×g for 1 minute, and, after removing the supernatant, resuspended in the thrombin solution. Subsequently, the thrombin solution in which the cells were suspended and the Hep/Col fibrinogen-added medium were mixed at 1:2 (volume ratio), and 50 μL of the mixed solution was laminated in the cell culture insert containing fibrin gel in which the cancer cells were seeded obtained as above. Then, the cell culture insert containing fibrin gel in which the cancer cells were seed as described above was incubated in a CO₂ incubator (37° C., 5% CO₂) for 7 days. During the culture, the medium was exchanged as appropriate.

### Tissue Section Analysis

On day 1 and day 8 of culture of cell aggregate, thin slices were prepared in the same manner as in Experimental Example 2 and subjected to HE staining and IHC staining, and the positions of cancer cells in the HE stained and IHC stained three-dimensional cellular tissues were observed.

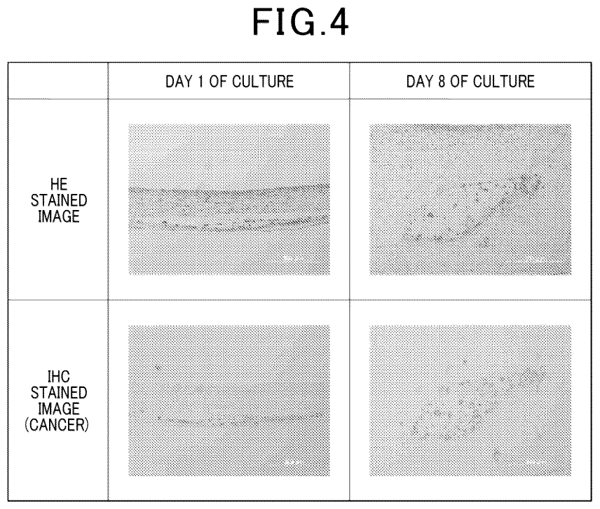

FIG. 4 is representative micrographs of three-dimensional cellular tissue fragments on day 1 and day 8 of culture. As shown in FIG. 4, even when the cancer cells were present in the stromal cells, the cancer cells on day 8 of culture were found not to have migrated from the positions on day 1 of culture on the stromal cells.

In recent years, the superiority of using three-dimensional cellular tissues organized three-dimensionally over cells grown on a flat plate has been shown in the fields of regenerative medicine and assay systems for drugs that require an environment close to that of a living body. Accordingly, various techniques for constructing three-dimensional cellular tissues in vitro have been developed.

The inventors of the present invention have previously developed a technique for producing three-dimensional cellular tissues, including obtaining a mixture containing cells suspended in a solution containing at least a cationic buffer solution, an extracellular matrix component and a polyelectrolyte, collecting the cells from the obtained mixture to form cell aggregates on a substrate, and culturing the cells to obtain three-dimensional cellular tissues (e.g., see JP 6639634 B). Three-dimensional cellular tissues can be used for, for example, biological tissue models or solid cancer models for use in various assays such as drug screening.

In order to use a three-dimensional cellular tissue as a biological tissue model, a solid cancer model, or the like for use in an assay such as drug screening, it is desired to construct a model more closely resembling a biological tissue. When a three-dimensional cellular tissue is prepared by a conventional method for inserting target cells such as cancer cells into the three-dimensional cellular tissue, the target cells can be inserted as a two-dimensional layer into the three-dimensional cellular tissue. However, it is difficult to culture the target cells while maintaining them at predetermined positions in the three-dimensional cellular tissue.

Therefore, an embodiment of the present invention provides a technique for culturing target cells while maintaining them at predetermined positions in a three-dimensional cellular tissue.

The present invention includes the following aspects.

1. A method of producing three-dimensional cellular tissues includes: obtaining a stromal cell-containing mixture containing stromal cells, a cationic substance, an extracellular matrix component and a polyelectrolyte, or a stromal cell-containing mixture containing stromal cells, a cationic substance and a fragmented extracellular matrix component; gelling the stromal cell-containing mixture to obtain a first gel composition containing stromal cells; obtaining a target cell-containing mixture containing target cells, a cationic substance, an extracellular matrix component and a polyelectrolyte, or a target cell-containing mixture containing target cells, a cationic substance and a fragmented extracellular matrix component; placing the target cell-containing mixture in contact with the first gel composition; gelling the target cell-containing mixture to obtain a second gel composition containing target cells; and incubating the first gel composition and the second gel composition to obtain three-dimensional cellular tissues.

2. A method of producing three-dimensional cellular tissues includes: obtaining a stromal cell-containing mixture containing stromal cells, a cationic substance, an extracellular matrix component and a polyelectrolyte; gelling the stromal cell-containing mixture to obtain a first gel composition containing stromal cells; obtaining a target cell-containing mixture containing target cells, a cationic substance, an extracellular matrix component and a polyelectrolyte; placing the target cell-containing mixture in contact with the first gel composition; gelling the target cell-containing mixture to obtain a second gel composition containing target cells; and incubating the first gel composition and the second gel composition to obtain three-dimensional cellular tissues.

3. A method of producing three-dimensional cellular tissues includes: obtaining a stromal cell-containing mixture containing stromal cells, a cationic substance, and a fragmented extracellular matrix component; gelling the stromal cell-containing mixture to obtain a first gel composition containing stromal cells; obtaining a target cell-containing mixture containing target cells, a cationic substance, and a fragmented extracellular matrix component; placing the target cell-containing mixture in contact with the first gel composition; gelling the target cell-containing mixture to obtain a second gel composition containing target cells; and incubating the first gel composition and the second gel composition to obtain three-dimensional cellular tissues.

4. The production method according to any one of 1 to 3 further includes: after gelling the target cell-containing mixture to obtain the second gel composition containing target cells, laminating the stromal cell-containing mixture on the second gel composition and gelling the stromal cell-containing mixture to form a third gel composition containing stromal cells on the second gel composition.

5. In the production method according to any one of 1 to 4, the first gel composition includes a first gel component in which at least one selected from the extracellular matrix component, agarose, pectin and fibrin monomers is gelled.

6. In the production method according to 5, the first gel component is a fibrin gel in which fibrin monomers are gelled, and the obtaining of the first gel composition containing stromal cells includes mixing thrombin and fibrinogen with the stromal cell-containing mixture.

7. In the production method according to any one of 1 to 6, the second gel composition includes a second gel component in which at least one selected from the extracellular matrix component, agarose, pectin and fibrin monomers is gelled.

8. In the production method according to 7, the second gel component is a fibrin gel in which fibrin monomers are gelled, and the obtaining of the second gel composition containing target cells includes mixing thrombin and fibrinogen with the target cell-containing mixture.

9. In the production method according to 6, the obtaining of the first gel composition containing stromal cells includes: adding thrombin to the stromal cell-containing mixture; and adding fibrinogen to the stromal cell-containing mixture to which thrombin has been added, whereby a fibrin gel is formed to gel the stromal cell-containing mixture.

10. In the production method according to 8, the obtaining of the second gel composition containing target cells includes: adding thrombin to the target cell-containing mixture; and adding fibrinogen to the target cell-containing mixture to which thrombin has been added, whereby a fibrin gel is formed to gel the target cell-containing mixture.

11. In the production method according to any one of 1 to 10, the extracellular matrix component is selected from collagen, laminin, fibronectin, vitronectin, elastin, tenascin, entactin, fibrin, proteoglycan, and combinations thereof.

12. In the production method according to any one of 1 to 11, a total content of the extracellular matrix component in the stromal cell-containing mixture or the target cell-containing mixture is 0.005 mg/mL or more and 1.5 mg/mL or less.

13. In the production method according to any one of 1 to 12, the polyelectrolyte is selected from glycosaminoglycan, dextran sulfate, rhamnan sulfate, fucoidan, carrageenan, polystyrene sulfonic acid, polyacrylamide-2-methylpropanesulfonic acid, polyacrylic acid, and combinations thereof.

14. In the production method according to any one of 1 to 13, a total content of the polyelectrolyte in the stromal cell-containing mixture or the target cell-containing mixture is 0.005 mg/mL or more.

15. In the production method according to any one of 1 to 14, the fragmented extracellular matrix component is fragmented collagen.

16. The production method according to any one of 1 to 15 further includes: before obtaining the first gel composition containing stromal cells, applying an external force to the stromal cell-containing mixture to obtain a stromal cell aggregate; and obtaining the first gel composition containing stromal cells, gelling, as the stromal cell-containing mixture, the stromal cell aggregate to obtain the first gel composition containing stromal cells.

17. The production method according to any one of 1 to 16 further includes, before placing the target cell-containing mixture in contact with the first gel composition, applying an external force to the target cell-containing mixture to obtain a target cell aggregate; in the placing of the target cell-containing mixture in contact with the first gel composition, placing, as the target cell-containing mixture, the target cell aggregate in contact with the first gel composition; and in the obtaining of the second gel composition containing target cells, gelling, as the target cell-containing mixture, the target cell aggregate to obtain the second gel composition containing target cells.

18. In the production method according to any one of 1 to 17, the target cells are cancer cells.

19. In a three-dimensional cellular tissue, a target cell gel section containing target cells, a cationic substance, an extracellular matrix component, a polyelectrolyte and a second gel component, or a target cell gel section containing target cells, a cationic substance, a fragmented extracellular matrix component and a second gel component is placed in contact with a stromal cell gel section containing stromal cells, a cationic substance, an extracellular matrix component, a polyelectrolyte and a first gel component, or a stromal cell gel section containing stromal cells, a cationic substance, a fragmented extracellular matrix component and a first gel component.

20. In a three-dimensional cellular tissue, a target cell gel section containing target cells, a cationic substance, an extracellular matrix component, a polyelectrolyte and a second gel component is placed in contact with a stromal cell gel section containing stromal cells, a cationic substance, an extracellular matrix component, a polyelectrolyte and a first gel component.

21. In a three-dimensional cellular tissue, a target cell gel section containing target cells, a cationic substance, a fragmented extracellular matrix component and a second gel component is placed in contact with a stromal cell gel section containing stromal cells, a cationic substance, a fragmented extracellular matrix component and a first gel component.

22. In a three-dimensional cellular tissue according to any one of 19 to 21, the target cells are cancer cells.

Obviously, numerous modifications and variations of the present invention are possible in light of the above teachings. It is therefore to be understood that within the scope of the appended claims, the invention may be practiced otherwise than as specifically described herein.

1. A method of producing three-dimensional cellular tissues, comprising:
obtaining a stromal cell-containing mixture comprising stromal cells, a cationic substance, an extracellular matrix component and a polyelectrolyte, or a stromal cell-containing mixture comprising stromal cells, a cationic substance and a fragmented extracellular matrix component;
gelling the stromal cell-containing mixture to obtain a first gel composition comprising stromal cells;
obtaining a target cell-containing mixture comprising target cells, a cationic substance, an extracellular matrix component and a polyelectrolyte, or a target cell-containing mixture comprising target cells, a cationic substance and a fragmented extracellular matrix component;
placing the target cell-containing mixture in contact with the first gel composition;
gelling the target cell-containing mixture to obtain a second gel composition comprising target cells; and
incubating the first gel composition and the second gel composition to obtain three-dimensional cellular tissues.

2. The production method according to claim 1, further comprising:
after gelling the target cell-containing mixture to obtain the second gel composition comprising target cells, laminating the stromal cell-containing mixture on the second gel composition and gelling the stromal cell-containing mixture to laminate a third gel composition comprising stromal cells on the second gel composition.

3. The production method according to claim 1, wherein the first gel composition includes a first gel component in which at least one selected from the group consisting of the extracellular matrix component, agarose, pectin and fibrin monomers is gelled.

4. The production method according to claim 3, wherein the first gel component is a fibrin gel in which fibrin monomers are gelled, and the obtaining of the first gel composition comprising stromal cells includes mixing thrombin and fibrinogen with the stromal cell-containing mixture.

5. The production method according to claim 1, wherein the second gel composition includes a second gel component in which at least one selected from the group consisting of the extracellular matrix component, agarose, pectin and fibrin monomers is gelled.

6. The production method according to claim 5, wherein the second gel component is a fibrin gel in which fibrin monomers are gelled, and the obtaining of the second gel composition comprising target cells includes mixing thrombin and fibrinogen with the target cell-containing mixture.

7. The production method according to claim 4, wherein the obtaining of the first gel composition comprising stromal cells includes: adding thrombin to the stromal cell-containing mixture; and adding fibrinogen to the stromal cell-containing mixture to which thrombin has been added, whereby a fibrin gel is formed to gel the stromal cell-containing mixture.

8. The production method according to claim 6, wherein the obtaining of the second gel composition comprising target cells includes: adding thrombin to the target cell-containing mixture; and adding fibrinogen to the target cell-containing mixture to which thrombin has been added, whereby a fibrin gel is formed to gel the target cell-containing mixture.

9. The production method according to claim 1, wherein the extracellular matrix component is selected from the group consisting of collagen, laminin, fibronectin, vitronectin, elastin, tenascin, entactin, fibrin, proteoglycan, and combinations thereof.

10. The production method according to claim 1, wherein a total content of the extracellular matrix component in the stromal cell-containing mixture or the target cell-containing mixture is 0.005 mg/mL or more and 1.5 mg/mL or less.

11. The production method according to claim 1, wherein the polyelectrolyte is selected from the group consisting of glycosaminoglycan, dextran sulfate, rhamnan sulfate, fucoidan, carrageenan, polystyrene sulfonic acid, polyacrylamide-2-methylpropanesulfonic acid, polyacrylic acid, and combinations thereof.

12. The production method according to claim 1, wherein a total content of the polyelectrolyte in the stromal cell-containing mixture or the target cell-containing mixture is 0.005 mg/mL or more.

13. The production method according to claim 1, wherein the fragmented extracellular matrix component is fragmented collagen.

14. The production method according to claim 1, further comprising:
before obtaining the first gel composition comprising stromal cells, applying an external force to the stromal cell-containing mixture to obtain a stromal cell aggregate; and
in the obtaining of the first gel composition comprising stromal cells, gelling, as the stromal cell-containing mixture, the stromal cell aggregate to obtain the first gel composition comprising stromal cells.

15. The production method according to claim 1, further comprising:
before placing the target cell-containing mixture in contact with the first gel composition, applying an external force to the target cell-containing mixture to obtain a target cell aggregate;
in the placing of the target cell-containing mixture in contact with the first gel composition, placing, as the target cell-containing mixture, the target cell aggregate in contact with the first gel composition; and
in the obtaining of the second gel composition containing target cells, gelling, as the target cell-containing mixture, the target cell aggregate to obtain the second gel composition containing target cells.

16. The production method according to claim 1, wherein the target cells are cancer cells.

17. The production method according to claim 2, wherein the first gel composition includes a first gel component in which at least one selected from the group consisting of the extracellular matrix component, agarose, pectin and fibrin monomers is gelled.

18. The production method according to claim 17, wherein the first gel component is a fibrin gel in which fibrin monomers are gelled, and the obtaining of the first gel composition comprising stromal cells includes mixing thrombin and fibrinogen with the stromal cell-containing mixture.

19. A three-dimensional cellular tissue, wherein a target cell gel section comprising target cells, a cationic substance, an extracellular matrix component, a polyelectrolyte and a second gel component, or a target cell gel section comprising target cells, a cationic substance, a fragmented extracellular matrix component and a second gel component is placed in contact with a stromal cell gel section comprising stromal cells, a cationic substance, an extracellular matrix component, a polyelectrolyte and a first gel component, or a stromal cell gel section comprising stromal cells, a cationic substance, a fragmented extracellular matrix component and a first gel component.

20. The three-dimensional cellular tissue according to claim 19, wherein the target cells are cancer cells.
